SEQUENCE LISTING (1 sequence)

SEQ ID NO: 1 - protein, 906 aa, Homo sapiens
MCRIAGALRTLLPLLAALLQASVEASGEIALCKTGFPEDVYSAVLSKDVHEGQPLLNVKFSNCNGKRKVQYESSEPADFKVDEDGMVYAVRSFPLSSEHAKFLIYAQDKETQEKWQVAVKLSLKPTLTEESVKESAEVEEIVFPRQFSKHSGHLQRQKRDWVIPPINLPENSRGPFPQELVRIRSDRDKNLSLRYSVTGPGADQPPTGIFIINPISGQLSVTKPLDREQIARFHLRAHAVDINGNQVENPIDIVINVIDMNDNRPEFLHQVWNGTVPEGSKPGTYVMTVTAIDADDPNALNGMLRYRIVSQAPSTPSPNMFTINNETGDIITVAAGLDREKVQQYTLIIQATDMEGNPTYGLSNTATAVITVTDVNDNPPEFTAMTFYGEVPENRVDIIVANLTVTDKDQPHTPAWNAVYRISGGDPTGRFAIQTDPNSNDGLVTVVKPIDFETNRMFVLTVAAENQVPLAKGIQHPPQSTATVSVTVIDVNENPYFAPNPKIIRQEEGLHAGTMLTTFTAQDPDRYMQQNIRYTKLSDPANWLKIDPVNGQITTIAVLDRESPNVKNNIYNATFLASDNGIPPMSGTGTLQIYLLDINDNAPQVLPQEAETCETPDPNSINITALDYDIDPNAGPFAFDLPLSPVTIKRNWTITRLNGDFAQLNLKIKFLEAGIYEVPIIITDSGNPPKSNISILRVKVCQCDSNGDCTDVDRIVGAGLGTGAIIAILLCIIILLILVLMFVVWMKRRDKERQAKQLLIDPEDDVRDNILKYDEEGGGEEDQDYDLSQLQQPDTVEPDAIKPVGIRRMDERPIHAEPQYPVRSAAPHPGDIGDFINEGLKAADNDPTAPPYDSLLVFDYEGSGSTAGSLSSLNSSSSGGEQDYDYLNDWGPRFKKLADMYGGGDD

(19) United States
(12) Patent Application Publication
(10) Pub. No.: US 2015/0050240 A1
(43) Pub. Date: Feb. 19, 2015

(54) TREATMENT OF FIBROSIS

(71) Applicant: NOVARTIS AG, Basel (CH)

(72) Inventors: Irena Rumenova Konstantinova, Horsham (GB); Andrew Christopher Pearce, Compton Dando (GB)

(73) Assignee: NOVARTIS AG, Basel (CH)

(21) Appl. No.: 14/388,091

(22) PCT Filed: Mar. 13, 2013
(86) PCT No.: PCT/IB2013/051989

§ 371 Date: Sep. 25, 2014

Related U.S. Application Data
(60) Provisional application No. 61/616,023, filed on Mar. 27, 2012.

(51) Int. Cl. C07K 16/28; A61K 39/395; A61K 38/21; A61K 38/12; A61K 31/4412

(52) U.S. Cl. CPC C07K 16/28; A61K 38/12; A61K 31/4412; A61K 38/21; A61K 39/3955
USPC 424/85.4; 424/130.1; 514/21.1

(57) ABSTRACT

This invention is in the field of treatment of fibrosis. In particular, it relates to the treatment of IPF using N-Cadherin antibodies. The antibody may be any antagonising or neutralizing N-Cadherin antibody suitable for therapeutic use.

### TECHNICAL FIELD

This invention is in the field of treatment of fibrosis using an antagonist of N-Cadherin. In particular, it relates to the treatment of Idiopathic Pulmonary fibrosis (IPF) using N-Cadherin antibodies. Such an antibody may be any antagonising or neutralizing N-Cadherin antibody suitable for therapeutic use.

### BACKGROUND

### Lung Fibrosis—Pathology and Etiology

Idiopathic pulmonary fibrosis (IPF) is the most common form of interstitial lung disease. It is a progressive, terminal condition affecting 5 million patients worldwide (Gharaee-Kermani and Phan, 2005; Meltzer and Noble, 2008). Long-term survival of IPF patients is poor with a 5 year survival rate of only 20% (Scotton and Chambers, 2007). The pathology of IPF is characterised by excessive deposition and accumulation of disorganised collagen fibres and other extracellular matrix components, resulting in stiffening of the tissue, loss of flexibility and progressive decline in lung function. The prognosis of IPF has been compared to that of many malignancies with a median survival of only 2 to 3 years following diagnosis (Scotton and Chambers, 2007). Despite more than 50 years of investigation, to date there is no efficacious therapy for IPF with lung transplantation being the only measure shown to prolong survival (Walter et al., 2006), and patients are progressing to a fatal outcome in a few years after diagnosis. IPF has been considered to be mainly inflammatory process and therefore historically the natural choice of treatment has been the use of corticosteroids and other immunosuppressive drugs (Douglas et al., 2000; Selman et al., 1998). However, current therapies based on altering the inflammatory component are only marginally effective, there is no evidence of long-term improvement and they have serious side effects (Mapel et al., 1996; Selman et al., 2001).

Therapy directed against fibroblasts proliferation and extracellular matrix accumulation is now being evaluated. Two such agents are pirfenidone and interferons. Pirfenidone reduces collagen synthesis and blocks mitogenic effects of pro-fibrotic cytokines in lung fibroblasts from IPF patients (Kaneko et al., 1998; Raghu et al., 1999). In a phase II trial with IPF patients, survival rates were improved, pulmonary functional tests stabilized or improved in some patients and there were no severe side effects. However, chest radiographs did not improve, the data was difficult to interpret and additional studies were required to assess efficacy (Raghu et al., 1999). The results from a phase III clinical trial indicated that Pirfenidone might positively affect lung function in IPF patients (Bouros, 2011) and recently it was approved for treatment in Europe, Japan and India, although its precise mechanism of action is unclear. FDA requested additional clinical trials to evaluate Pirfenidone efficacy before approval.

Despite similar encouraging in vitro studies, an interferon-based clinical trial with IPF patients, refractory to corticosteroid or immunosuppressive therapy, showed no improvement. In another study with small cohort IPF patients, there was a favourable response to interferon gamma in a combination with prednisone (Ziesche et al., 1999). A more recent clinical trial with interferon was discontinued in 2007 due to ineffectiveness and slightly increased patients death rates compared with the patients receiving placebo. At the moment, Pirfenidone is the only agent prescribed for IPF, which might delay lung decline and improve lung function.

Hence, there remains an unmet medical need for therapies for treatment of fibrosis in general and IPF in particular.

### SUMMARY OF THE INVENTION

It has now been found that N-Cadherin mediated signalling contributes to fibrosis in subjects suffering from IPF. The invention therefore provides an N-Cadherin antagonist which inhibits or neutralizes the activity of N-Cadherin for use in the treatment or prevention of fibrosis. In one embodiment the N-Cadherin antagonist is an anti-N-Cadherin antibody. In another embodiment the N-Cadherin antibody is for use in the treatment or prevention of Idiopathic Pulmonary Fibrosis.

The invention also provides for the use of an N-Cadherin antagonist which inhibits or neutralizes the activity of N-Cadherin in the manufacture of a medicament for the treatment or prevention of fibrosis. In one embodiment an anti-N-Cadherin antibody is used. In another embodiment the use is in the manufacture of a medicament for treatment or prevention of Idiopathic Pulmonary Fibrosis.

The invention also provides for a method of treating or preventing fibrosis comprising of administering a N-Cadherin antagonist which inhibits or neutralizes the activity of N-Cadherin to a subject in need thereof. In one embodiment the method comprises the administration of an anti-N-Cadherin antibody. In another embodiment the method is for the treatment or prevention of Idiopathic Pulmonary Fibrosis.

The invention also provides the antibody, use or method wherein the antibody is formulated with a pharmaceutically acceptable carrier.

The invention also provides the antibody, use or method wherein the antibody is co-administered sequentially or simultaneously with pirfenidone or interferons.

### LIST OF FIGURES

FIG. 1. Cell-cell contacts between human lung fibroblasts induce FMT. A. Western blots for SMA, N-Cadherin and OB-Cadherin from HLFs seeded at low cell density (LCD) and high cell density (HCD) in the absence or presence of TGFβ. B. Co-staining for N-Cadherin and SMA in HLF culture. The squared region in the merged is zoomed in to demonstrate the co-staining.

FIG. 2. N-Cadherin is involved in human lung fibroblast-to-myofibroblast transition in vitro. A. Western blots for SMA and β-actin from HLFs seeded in the absence or presence of TGFβ, stimulated with an Fc control or N-Cadherin-Fc. B. N-cadherin knockdown in HLFs using negative control siRNA and N-Cadherin siRNA measured by RT-PCR, and a Western blot for SMA in the siRNAs-transfected HLFs. C. Western blot for SMA from HLFs in the absence or presence of TGFβ treated with mouse IgG or N-Cadherin blocking antibody (GC-4).

FIG. 3. N-Cadherin knockdown inhibits pro-fibrotic functional effects in human lung fibroblasts. A. Effect of N-Cadherin gain and loss of function on HLF migration using N-Cadherin-Fc or N-Cadherin siRNA, respectively. B. Effect of N-Cadherin knockdown on serum-induced and growth factor induced HLF proliferation. Negative control siRNA effects are represented by the black bars and N-Cadherin siRNA effects—by the white bars. C. Effect of N-Cadherin knockdown on collagen secretion from HLFs. * represents statistical significance with a p value <0.05 and **—with a p value <0.001.

FIG. 4. N-Cadherin downstream signalling in human lung fibroblasts. A. Western blots for p-Ser552 β-catenin and total β-catenin from HLFs in the absence or presence of TGFβ. B. Western blots for p-Ser552 β-catenin and total β-catenin from HLFs transfected with negative siRNA and N-Cadherin siRNA.

FIG. 5. N- to OB-Cadherin switch in human lung fibroblasts. A. OB-cadheirn knockdown in HLFs using negative control siRNA and OB-Cadherin siRNA measured by RT-PCR. B. Western blots for SMA and N-Cadherin from negative siRNA and OB-Cadherin siRNA transfected HLFs in the absence or presence of TGFβ. C. Effect of OB-Cadherin loss of function on HLF migration using OB-Cadherin siRNA.

FIG. 6. Markers for epithelial-to-mesenchymal transition. EMT markers N-Cadherin, vimentin and E-Cadherin levels in A549 cells in the absence or presence of TGFβ or TGFβ+IL-1β, measured by RT-PCR.

FIG. 7. N-Cadherin loss of function prevents EMT. A. Western blots for N-Cadherin, vimentin and E-Cadherin from A549 cells and HBECs in the absence or presence of TGFβ. B. Western blots for N-Cadherin and vimentin from A549 cells transfected with negative control siRNA or N-Cadherin siRNA in the absence or presence of TGFβ. C. Effect of N-Cadherin gain of function on EMT using N-Cadherin-Fc. D. Effect of N-Cadherin loss of function on EMT using N-Cadherin neutralising antibody.

FIG. 8. N-Cadherin expression in animal models of IPF and in IPF patient derived lung fibroblasts. A. N-Cadherin immunohistochemistry staining of mouse lung sections from day 14 in a time course experiment using the asbestos model. PBS and TitO2 were used as negative controls. B. N-Cadherin expression levels in IPF patients derived HLFs compared with non-IPF patient derived (healthy) HLFs measured by Western blot and quantified using optical density measurement.

FIG. 9. N-cadherin expression in IPF patients lung tissue. N-Cadherin expression levels in IPF patients lung biopsies compared with non-IPF patients (healthy) lung biopsies measured by Western blot and quantified using optical density measurement. Each number above the SDS-PAGE image represents a different patient. The data for each of the healthy donors was consolidated. RUL, right upper lobe; RML, right middle lobe; RLL, right lower lobe; LUL, left upper lobe; LML, left middle lobe; LLL, left lower lobe.

### DESCRIPTION OF THE INVENTION

The initiating event of IPF is largely unknown though is believed to represent aberrant activation of normal repair pathways. During normal wound healing damaged cells at the site of injury cause a local elevation in pro-inflammatory cytokines leading to recruitment of inflammatory cells to clear infection and fibroblasts to repair the damaged tissue. Local upregulation of growth factors combined with cellular differentiation and activation results in tissue remodelling around the wound area. Finally, the inflammation is resolved and the repair processes are deactivated (Hinz et al., 2007; Tomasek et al., 2002b; Werner and Grose, 2003). Fibrosis is thought to be caused due to development of chronicity of the inflammation phase or repair pathway activation, or a failure of resolution (Follonier et al., 2008; Gharaee-Kermani and Phan, 2005). IPF shares many similarities with fibrosis in other tissues including skin, liver and kidney (Border and Noble, 1994). However, in the majority of cases lung fibrosis is not co-diagnosed with fibrosis in other organs, demonstrating that the local environment plays a critical role in causing and sustaining fibrosis (Meltzer and Noble, 2008).

Fibroproliferative diseases, including pulmonary fibrosis, systemic sclerosis, liver cirrhosis and progressive kidney disease share common cellular fibrogenetic mechanisms and tissue remodelling abnormalities. Most fibrotic disorders have in common an irritant that sustains the production of growth factors, proteolytic enzymes, angiogenic factors, and fibrogenic cytokines including TGFβ, which together stimulate myofibroblasts differentiation, proliferation and deposition of connective tissue elements that progressively remodel and destroy normal tissue architecture (Tomasek et al., 2002a; Friedman, 2004; Chatziantoniou and Dussaule, 2005). Therefore, an N-Cadherin blocking agent interfering with the above processes could be used for the treatment not only of IPF but also of other fibrotic disorders.

### The Importance of Fibroblasts and Myofibroblasts in Fibrosis

Fibroblasts are cells of the mesenchyme lineage whose main function is to provide structural support for the tissues through maintenance of extracellular matrix homeostasis. Fibroblasts secrete the major component of the extracellular matrix, collagen. Consistent with the importance of excessive collagen production in fibrosis, fibroblasts are key cells in the progression of fibrotic disease and increased numbers are found in the lesions of fibrotic tissues (Hinz, 2007; Tomasek et al., 2002b). The fibroblasts in fibrotic lesions are believed to originate from 3 potential sources: Firstly, resident fibroblasts proliferate and migrate to fibrotic foci (Zhang et al., 1994). Secondly, fibroblasts are believed to be formed by trans-differentiation of epithelial cells in a process known as epithelial-mesenchymal transition (EMT) (Kim et al., 2006; Willis et al., 2005) and thirdly, circulating mesenchymal progenitors called fibrocytes are believed to be recruited (Abe et al., 2001; Hinz, 2007; Phillips et al., 2004). The relative role of each of these processes in the development of IPF remains to be determined. In addition to the increase in number of fibroblasts the phenotype of the cells is altered. These cells are normally relatively quiescent, however at sites of fibrosis the fibroblasts are hyper-proliferative, hyper-secretory and pro-inflammatory, more motile and more contractile and are resistant to apoptosis (Horowitz et al., 2004; Horowitz et al., 2006; Raghu et al., 1988; Zhang and Phan, 1999; Zhang et al., 1994). One of the mechanisms for achieving these phenotypic changes is the trans-differentiation of fibroblasts into myofibroblasts in a process known as fibroblast-myofibroblast transition (FMT). Myofibroblasts are characterised by the expression of the contractile cytoskeleton protein smooth muscle actin. Smooth muscle actin provides greater mechanical strength leading to the cells being pro-migratory and pro-contractile (Hinz et al., 2003; Hinz and Gabbiani, 2003a; Tomasek et al., 2002b).

In addition, myofibroblasts are pro-proliferative and pro-secretory relative to quiescent fibroblasts (Sebe et al., 2008). During the normal wound healing process FMT is induced by secreted mediators such as TGFβ, the myofibroblasts migrate into the wound site where they mediate tissue remodelling and then undergo apoptosis. In IPF, myofibroblasts having the pro-fibrotic phenotypes described above, are highly enriched in fibrotic lesions and are found to be resistant to apoptosis (Horowitz et al., 2004; Vittal et al., 2005; Zhang and Phan, 1999).

### Cell-Cell Contacts in Wound Healing and Fibrosis

Intercellular adhesion is mediated by cell-cell contacts involving specific molecular complexes, which play specific roles in cell-cell communication (Gumbiner, 2005a; Gumbiner, 2005b; Nelson and Nusse, 2004). These junctional complexes define the strength and function of the cell-cell adhesion. One of the most common types of intercellular junctions is the adherens junction. Adherens junctions are characterised by the presence of calcium dependent adhesion molecules called Cadherins, which directly mediate the cell-cell contact via trans-cellular, homotypic interactions (Gumbiner, 2005b; Patel et al., 2003). The classical Cadherins, which include E- and N-Cadherin, show a high level of specificity in their interactions, binding preferentially to the same Cadherin isoform on a neighbouring cell and play an important role in cell recognition and tissue maintenance (Takeichi, 1991). Cadherins couple to multiple effects on RTK signalling, cytoskeleton rearrangements and gene expression by activating catenins and by direct interaction and crosstalk with RTKs (Nelson and Nusse, 2004). Different Cadherins lead to differential effects on signalling pathways and therefore bestow distinct effects on proliferative, migratory and other cellular responses (Gumbiner, 2005b; Takeichi, 1991). N-Cadherin is highly expressed in neurons, forms relatively weak, short-lived homophilic interactions and is thus associated with an increase in cell migration whereas E-Cadherin is expressed in epithelial cells, forms tight, longer-lived homophilic interactions and is thus associated with non-migration and maintenance of epithelial barrier function. OB-Cadherin is highly expressed in osteoblasts, forms tight and stable adhesions and couples to pro-proliferative signalling pathways to strengthen bone in response to bone atrophy or damage (Nelson and Nusse, 2004; Williams et al., 1994). Switching of Cadherin expression is associated with the gain- and loss-of these cellular characteristics and these so called “Cadherin-switches” define key stages during development and in functional transitions in adult tissues. For example, such switches are the transition of E-Cadherin to N-Cadherin in the different stages of development and the transition of VE-Cadherin to N-Cadherin in endothelial cells when plasticity of the vascular system is required during angiogenesis (Luo and Radice, 2005; Takeichi, 1991; Thiery et al., 2009). In addition, Cadherins have been linked to the pathology of a number of diseases. OB-Cadherin is associated with the bone and joint remodelling in rheumatoid arthritis (Farina et al., 2009). The switch from E-Cadherin to N-Cadherin in epithelial cells is associated with EMT that leads to increased invasiveness and metastasis in many tumours (Cavallaro et al., 2002; Thiery et al., 2009) and the switch from VE-Cadherin to N-Cadherin is associated with the angiogenesis in tumour progression (Blaschuk and Rowlands, 2000; Luo and Radice, 2005). It has previously been shown that intercellular communication plays a crucial role in tissue repair during wound healing (Follonier et al., 2008; Scotton and Chambers, 2007).

The increased density of activated myofibroblasts in the wound leads to an increase in the number of cell-cell contacts and the formation of a mechanical, force-generating intercellular network that contracts the extracellular matrix, closing the wound. In healthy lung, fibroblasts are scattered throughout the extracellular matrix regulating the balance between deposition and degradation of matrix proteins. Therefore, under normal conditions fibroblasts are rarely in proximity long enough to form cell-cell contacts. Adherens junctions are absent in normal tissue fibroblasts, which do not develop stress fibres (Welch et al., 1990). A previous report has suggested that myofibroblast differentiation is accompanied by the formation of adherens junctions, coordinating the contractility of the cells (Hinz et al., 2003; Hinz and Gabbiani, 2003b; Hinz et al., 2004). A further study demonstrated that unlike normal fibroblasts, which do not form adherens junctions, myofibroblasts display significantly higher contraction coordination (Follonier et al., 2008).

### Preclinical Models of Fibrosis

Several animal models have been developed to study lung fibrosis and have been used to identify key cells, molecules and processes likely to be involved in the development of human IPF. Each of these model systems has advantages and disadvantages but none of them recapitulates clearly and fully the mechanisms and manifestations of the human disease (Moore and Hogaboam, 2008).

The bleomycin model of IPF is the most commonly used and the best characterised murine model. Administration of this antibiotic results in pulmonary toxicity, lung injury and fibrosis in large number of animals—mice, rats, guinea pigs, rabbits, dogs and primates. The initial pathology affects the lung endothelium, which allows access of the drug to the alveoli where pathological response includes damage to the alveolar epithelium, leakage of liquid and plasma in the alveolar space, necrosis of alveolar cells type I and metaplasia of alveolar cells type II. Inflammatory and mesenchymal cells infiltration, altered epithelial-mesencymal interactions and signs of fibrosis are noted in the subpleural regions. Subsequently the inflammatory cells are cleared, fibroblasts proliferate and synthesise extracellular matrix (Phan et al., 1980; Thrall et al., 1979). The accumulated collagen is measured as a hallmark of the resulting fibrosis (Muggia et al., 1983). A disadvantage of this model is that fibrosis does not develop in all animals, the process is strain dependent (Schrier et al., 1983) and the fibrosis is self-limited and resolves instead of being slow progressing and irreversible (Phan et al., 1983). Therefore, one of the most critical characteristics of human IPF is not present in this model.

Administration of mineral fibres in the rodent lungs results in development of fibrotic regions resembling lesions formed in humans following occupational exposure to mineral dust and aerosols. This was used to develop the asbestos model of IPF. The response in this model is characterised by fibrotic regions and inflammatory infiltration in the bronchoalveolar lavage fluid. Asbestos is not cleared from the lungs and therefore the fibrotic stimulus is persistent (Davis et al., 1998b; Davis et al., 1998a). However, the response is strain dependent and anti-inflammatory therapies have no effect (Barbarin et al., 2005).

Other models include FITC-induced, irradiation-induced fibrosis models and virus targeted transgenic models. FITC instillation results in inflammatory infiltration and epithelial hyperplasia, but fibrotic changes have been noted only in the regions of FITC deposition (Roberts et al., 1995). The irradiation model is strain dependent (Sharplin and Franko, 1989). Viral transgenic agent induced-fibrosis is not persistent and affects predominantly epithelial cells (Engelhardt et al., 1994).

In summary, animal models of IPF represent good research tools for studying and validating cells, mediators and processes likely to contribute to the disease but are not established as predictive preclinical models for the human disease and major discrepancies between drug effects in animal models and in clinical trials have recently been described (Moeller et al., 2008; Perel et al., 2007).

In the context of the present application the best predictive data is therefore obtained by in vitro experiments with primary disease-relevant human lung fibroblasts (HLFs) from both, healthy donors and IPF patients, as well as with an alveolar type II cell line (A549). These include gene of interest expression level comparison, loss and gain of function effects on FMT, proliferation, migration and survival in HLFs and EMT in A549 cells. Similar functional data are widely used in the scientific literature and in drug discovery to predict anti-fibrotic effects of potential IPF targets (King, Jr. et al., 2011; Moeller et al., 2008; Selman et al., 2001).

Using these methods (see examples), we have demonstrated that intercellular contacts are formed in human lung fibroblasts (HLFs) in vitro when cultured at high cell density. These cells express N-Cadherin and this expression correlates with SMA expression. We demonstrate that N-Cadherin plays a role in regulating fibroblast-to-myofibroblast transition, collagen secretion, fibroblast proliferation and migration and a PI3-K, Akt-dependent survival pathway. Furthermore, N-Cadherin expression was increased during epithelial-mesenchymal transition induced by TGFβ and IL-1β, and siRNA to N-Cadherin inhibited EMT. N-Cadherin was found to be upregulated in the asbestos preclinical mouse model of IPF and in fibroblasts from the lungs of IPF patients. Together these data demonstrate a novel role for N-Cadherin in fibroblast-myofibroblast transition, fibroblast function and epithelial-mesenchymal transition and suggest that N-Cadherin and cell-cell adhesion regulate the development of a fibrotic phenotype. N-Cadherin is an important novel target for the treatment of IPF and other fibrotic disorders.

These findings are a clear indication that N-Cadherin is involved in driving the tissue remodeling that accompanies fibrosis (IPF), and that anti N-Cadherin therapy will be a useful treatment for subjects suffering from IPF or other fibroproliferative diseases.

The human mature N-Cadherin polypeptide has the below sequence. The extracelluar domain is underlined.

MCRIAGALRTLLPLLAALLQASVEASGEIALCKTGFPEDVYSAVLSKDV
HEGQPLLNVKFSNCNGKRKVQYESSEPADFKVDEDGMVYAVRSFPLSSE
HAKFLIYAQDKETQEKWQVAVKLSLKPTLTEESVKESAEVEEIVFPRQF
SKHSGHLQRQKRDWVIPPINLPENSRGPFPQELVRIRSDRDKNLSLRYS
VTGPGADQPPTGIFIINPISGQLSVTKPLDREQIARFHLRAHAVDINGN
QVENPIDIVINVIDMNDNRPEFLHQVWNGTVPEGSKPGTYVMTVTAIDA
DDPNALNGMLRYRIVSQAPSTPSPNMFTINNETGDIITVAAGLDREKVQ
QYTLIIQATDMEGNPTYGLSNTATAVITVTDVNDNPPEFTAMTFYGEVP
ENRVDIIVANLTVTDKDQPHTPAWNAVYRISGGDPTGRFAIQTDPNSND
GLVTVVKPIDFETNRMFVLTVAAENQVPLAKGIQHPPQSTATVSVTVID
VNENPYFAPNPKIIRQEEGLHAGTMLTTFTAQDPDRYMQQNIRYTKLSD
PANWLKIDPVNGQITTIAVLDRESPNVKNNIYNATFLASDNGIPPMSGT
GTLQIYLLDINDNAPQVLPQEAETCETPDPNSINITALDYDIDPNAGPF
AFDLPLSPVTIKRNWTITRLNGDFAQLNLKIKFLEAGIYEVPIIITDSG
NPPKSNISILRVKVCQCDSNGDCTDVDRIVGAGLGTGAIIAILLCIIIL
LILVLMFVVWMKRRDKERQAKQLLIDPEDDVRDNILKYDEEGGGEEDQD
YDLSQLQQPDTVEPDAIKPVGIRRMDERPIHAEPQYPVRSAAPHPGDIG
DFINEGLKAADNDPTAPPYDSLLVFDYEGSGSTAGSLSSLNSSSSGGEQ
DYDYLNDWGPRFKKLADMYGGGDD

### Antagonists Useful to Practice the Invention

In principle any antagonist, such as a LMW antagonist, a siRNA antagonist or Biologic therapeutic antagonist, such as for example an antibody, which inhibits or neutralizes the activity of N-Cadherin may be used in the invention. Examples of such antagonists useful to practice the present invention are disclosed in the following documents:

The first N-Cadherin antagonist tested for the treatment of cancer is ADH-1, a cyclic pentapeptide containing the histidine-alanin-valine (HAV) N-Cadherin extracellular domain recognition motif. Examples of N-Cadherin peptide antagonists for use in cancer treatment are described in WO2004048411, WO2004044000, US20090291967, WO2006116737, WO2009055937. A number of patent applications for LMW analogues of ADH-1 with improved potency have been filed but no clinical development has been reported (Burden-Gulley et al. 2009).

N-Cadherin antibodies for use in diagnosing, evaluating and treating cancer are described in WO2007109347, WO2009124281, WO2010054377 and WO2011119888. A recent paper describes the use of N-Cadherin antibodies in treating preclinical models of prostate cancer (Tanaka et al., 2010)). WO2011071543 discloses additional N-Cadherin inhibitory agents, for use in therapy of various non-fibrotic diseases.

The inhibition or neutralization of the activity of N-Cadherin in vitro can be assessed by measuring the pro-fibrotic functional effects (FMT, proliferation, collagen secretion, migration, survival etc), as described previously (Hinz et al., 2007; King, Jr. et al., 2011; Phan, 2002; Raghu et al., 2011; Scotton and Chambers, 2007; Zhang et al., 1994) and in this application. In preferred embodiments anti N-Cadherin antibodies (IgG, silenced IgG, Fab or other) of the invention inhibits a pro-fibrotic functional response with an IC₅₀ less than 10 nM, 5 nM, 2.5 nM, 1.0 nM, 0.5 nM, or less.

As used herein, the term “antibody” means a polypeptide comprising a framework region from an immunoglobulin gene or fragments thereof that specifically binds and recognizes an epitope, e.g. an epitope found on N-Cadherin, as described above. Thus, the term antibody includes whole antibodies (such as monoclonal, chimeric, humanised and human antibodies), including single-chain whole antibodies, and antigen-binding fragments thereof. The term “antibody” includes antigen-binding antibody fragments, including single-chain antibodies, which can comprise the variable regions alone, or in combination, with all or part of the following polypeptide elements: hinge region, CH₁, CH₂, and CH₃ domains of an antibody molecule. Also included within the definition are any combinations of variable regions and hinge region, CH₁, CH₂, and CH₃ domains. Antibody fragments include, e.g., but are not limited to, Fab, Fab′ and F(ab′)₂, Fd, single-chain Fvs (scFv), single-chain antibodies, disulphide-linked Fvs (sdFv) and fragments comprising either a V_{L} or V_{H} domain. Examples include: (i) a Fab fragment, a monovalent fragment consisting of the V_{L}, V_{H}, C_{L} and CH₁ domains; (ii) a F(ab′)₂ fragment, a bivalent fragment comprising two Fab fragments linked by a disulphide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341: 544-546, 1989; Muyldermans et al., TIBS 24: 230-235, 2001), which consists of a V_{H} domain; and (vi) an isolated complementarity determining region (CDR). The term “antibody” includes single domain antibodies, maxibodies, minibodies, intrabodies, diabodies, triabodies, tetrabodies, v-NAR and bis-scFv (see, e.g., Hollinger & Hudson, Nature Biotechnology, 23, 9, 1126-1136 (2005)). Antigen binding portions of antibodies can be grafted into scaffolds based on polypeptides such as Fibronectin type Ill (Fn3) (see U.S. Pat. No. 6,703,199, which describes fibronectin polypeptide monobodies). Antigen binding portions can be incorporated into single chain molecules comprising a pair of tandem Fv segments (VH—CH1-VH—CH1) which, together with complementary light chain polypeptides, form a pair of antigen binding regions (Zapata et al., Protein Eng. 8(10):1057-1062 (1995); and U.S. Pat. No. 5,641,870).

Preferably, the antibodies used in the invention bind specifically to N-Cadherin. Preferably, the antibodies used in the invention do not cross-react with an antigen other than N-Cadherin.

As used herein, an antibody that “specifically binds to N-Cadherin” is intended to refer to an antibody that binds to N-Cadherin with a K_{D} of 1×10⁻⁸ M or less, 1×10⁻⁹ M or less, or 1×10⁻¹° M or less. An antibody that “cross-reacts with an antigen other than N-Cadherin” is intended to refer to an antibody that binds that antigen with a K_{D} of 0.5×10⁻⁸ M or less, 5×10⁻⁹ M or less, or 2×10⁻⁹ M or less.

In an alternative embodiment, the antibody used in the invention is one which cross-blocks one or more of the antibodies recited above. By “cross-blocks” we mean an antibody which interferes with the binding of another antibody to N-Cadherin. Such interference can be detected, for example, using a competition assay using Biacore or ELISA. Such competition assays are described in WO2008/133722.

“Fibrosis”, “Fibrotic disease” or “Fibroproliferative disease” means the formation of excess fibrous connective tissue in a reparative process upon injury. Scarring is a result of continuous fibrosis that obliterates the affected organs or tissues architecture. As a result of abnormal reparative processes, which do not clear the formed scar tissue, fibrosis progresses further. Fibrosis can be found in various tissues, including the lungs, the liver, the skin and the kidneys. Examples of fibrosis include pulmonary fibrosis, liver cirrhosis, systemic sclerosis and progressive kidney disease.

“Idiopathic pulmonary fibrosis (IPF)” is a specific manifestation of idiopathic interstitial pneumonia (IIP), a type of interstitial lung disease. Interstitial lung disease, also known as diffuse parenchymal lung disease (DPLD), refers to a group of lung diseases affecting the interstitium. Microscopically, lung tissue from IPF patients shows a characteristic set of histological features known as usual interstitial pneumonia (UIP). UIP is therefore the pathologic presentation of IPF.

Other forms of IIPs include non-specific interstitial pneumonia (NSIP), desquamative interstitial pneumonia (DIP) and acute interstitial pneumonia (AIP). Examples of known causes of interstitial lung disease include sarcoidosis, hypersensitivity pneumonitis, pulmonary Langerhans cell histiocytosis, asbestosis and collagen vascular diseases such as scleroderma and rheumatoid arthritis.

The word “treatment” refers to a therapy for which the outcome is at least partial reversal of disease, i.e. at least partial reversal of fibrosis.

The word “prevention” refers to a therapy for which the outcome is a stop or at least a slowing down the progression of disease, i.e. at least a slowing down of fibrosis progression.

### Pharmaceutical Compositions

The antibodies used in the invention are generally formulated as a composition, e.g., a pharmaceutical composition, containing one or a combination of monoclonal antibodies, formulated together with a pharmaceutically acceptable carrier. For example, a pharmaceutical composition used in the invention can comprise a combination of antibodies that bind to different epitopes of N-Cadherin or that have complementary activities.

Pharmaceutical compositions used in the invention also can be administered in combination therapy, i.e., combined with other agents. For example, the combination therapy can include an anti-N-Cadherin antibody combined with pirfenidone or interferons. Such combinations may be administered simultaneously or sequentially. If administered sequentially, the period between administration of each agent may be a week or less, (e.g. a day or less, 12 hours or less, 6 hours or less, 1 hour or less, 30 minutes or less). The compositions are preferably formulated at physiological pH.

As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g., by injection or infusion). Depending on the route of administration, the active compound, i.e., antibody, immunoconjugate, or bispecific molecule, may be coated in a material to protect the compound from the action of acids and other natural conditions that may inactivate the compound.

Such pharmaceutical compositions may also include a pharmaceutically acceptable anti-oxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as, aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition. Prolonged absorption of the injectable compositions can be brought about by including in the composition an agent that delays absorption for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 per cent to about ninety-nine percent of active ingredient, from about 0.1 per cent to about 70 per cent, or from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals.

For administration of the antibody, the dosage ranges from about 0.0001 to about 100 mg/kg, and more usually about 0.01 to about 5 mg/kg, of the host body weight. For example dosages can be about 0.3 mg/kg body weight, about 1 mg/kg body weight, about 3 mg/kg body weight, about 5 mg/kg body weight, about 10 mg/kg body weight, about 20 mg/kg body weight, about 30 mg/kg body weight or within the range of about 1-about 30 mg/kg or about 1-about 10 mg/kg. An exemplary treatment regime entails administration about once per week, about once every two weeks, about once every three weeks, about once every four weeks, about once a month, about once every 3 months, about once every three to 6 months, about once every six months or about once a year. Dosage regimens for an anti-N-Cadherin antibody of the invention include about 1 mg/kg body weight or about 3 mg/kg body weight by intravenous administration, with the antibody being given using one of the following dosing schedules: about every four weeks for six dosages, then about every three months; about every three weeks; about 3 mg/kg body weight once followed by about 1 mg/kg body weight every three weeks.

In some methods, two or more monoclonal antibodies with different binding specificities are administered simultaneously or sequentially, in which case the dosage of each antibody administered falls within the ranges indicated. The combination could be an anti-N-Cadherin antibody combined with an anti-IL4 antibody. Antibody is usually administered on multiple occasions. Intervals between single dosages can be, for example, weekly, monthly, every three months, every six months or yearly. Intervals can also be irregular as indicated by measuring blood levels of antibody to the target antigen in the patient. In some methods, dosage is adjusted to achieve a plasma antibody concentration of about 1-about 1000 μg/ml and in some methods about 25-about 300 μg/ml.

Alternatively, antibody can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated or until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of an anti-N-Cadherin antibody of the invention can result in a decrease in severity of disease symptoms, an increase in frequency and duration of disease symptom-free periods, or a prevention of impairment or disability due to the disease affliction.

Compositions used in the present invention can be administered by one or more routes of administration using one or more of a variety of methods known in the art. As will be appreciated by the skilled artisan, the route and/or mode of administration will vary depending upon the desired results. Routes of administration for antibodies of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion. Intravenous and, subcutaneous administration are particularly preferred.

Alternatively, an antibody used in the invention can be administered by a nonparenteral route, such as a topical, epidermal or mucosal route of administration, for example, intranasally, orally, vaginally, rectally, sublingually or topically.

Inhaled administration is particularly preferred for the treatment of lung fibrosis, such as IPF.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in one embodiment, the compositions can be administered with a needleless hypodermic injection device, such as the devices shown in U.S. Pat. Nos. 5,399,163; 5,383,851; 5,312,335; 5,064,413; 4,941,880; 4,790,824 or 4,596,556. Examples of well known implants and modules useful in the present invention include: U.S. Pat. No. 4,487,603, which shows an implantable micro-infusion pump for dispensing medication at a controlled rate; U.S. Pat. No. 4,486,194, which shows a therapeutic device for administering medicants through the skin; U.S. Pat. No. 4,447,233, which shows a medication infusion pump for delivering medication at a precise infusion rate; U.S. Pat. No. 4,447,224, which shows a variable flow implantable infusion apparatus for continuous drug delivery; U.S. Pat. No. 4,439,196, which shows an osmotic drug delivery system having multi-chamber compartments; and U.S. Pat. No. 4,475,196, which shows an osmotic drug delivery system. These patents are incorporated herein by reference. Many other such implants, delivery systems, and modules are known to those skilled in the art.

The invention also provides a kit comprising a first component and a second component wherein the first component is an anti-N-Cadherin antibody or pharmaceutical composition as described above and the second component is instructions. In one embodiment, said instructions teach of the use of the antibody for treating fibrosis. The kit may further include a third component comprising one or more of the following: syringe or other delivery device, adjuvant, or pharmaceutically acceptable formulating solution.

General guidelines on the treatment of IPF, have been published by the American Thoracic Society, the European Respiratory Society and the British Thoracic Society (American Thoracic Society, 2000; B.T.SOCIETY and S.O.COMMITTEE, 1999; Raghu et al., 2011)

### General

The term “comprising” means “including” as well as “consisting” e.g. a composition “comprising” X may consist exclusively of X or may include something additional e.g. X+Y.

The term “about” in relation to a numerical value x means, for example, x±10%. References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of Current Protocols in Molecular Biology (F. M. Ausubel et al., eds., 1987) Supplement 30. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in Smith & Waterman (1981) Adv. Appl. Math. 2: 482-489

### EXAMPLES

### Materials and Methods

Cell Culture and siRNA Transfection Procedure

Primary human lung fibroblasts (Promocell) were maintained in DMEM supplemented with 10% heat-inactivated FBS, 1 mM Na-pyruvate and 1% Penicillin/Streptomycin and trypsinised every 4 days. Sub-confluent HLFs were electroporated via nucleofection (Amaxa electroporation system) with siRNAs.

IPF donors HLF (obtained from University of Michigan) were maintained in the same conditions.

A549 epithelial alveolar type II cells (ATCC) were maintained in DMEM supplemented with 10% heat-inactivated FBS and 1% Penicillin/Streptomycin and trypsinised every 4 days.

Sub-confluent cells were used for plating in all functional assays.

RT-PCR, siRNA Syntheses and Real-Time RT-PCR

Total RNA was extracted from HLFs, transcribed into cDNA and used for RT-PCR. Validated Taqman N-Cadherin, OB-Cadherin, vimentin or E-Cadherin primers and probes were purchased from Applied Biosystems.

Small interfering RNAs against N-Cadehrin and OB-Cadherin were purchased from Applied Biosystems. Knockdown efficiencies were tested by using real-time RT-PCR and confirmed to be 95% at the time of stimulation in the different functional assays.

### Confocal Light Microscopy

1:1000-diluted mouse-anti-N-Cadherin (BD Biosciences), rabbit-anti-OB-Cadherin (Santa Cruz), mouse-anti-SMA (Sigma) and rabbit-anti-β-catenin antibodies (Cell Signaling Technology) were used for staining of 4% PFA-fixed HLF cells or paraffin-embedded mouse lung sections from the asbestos model of IPF, described previously (De et al., 2004; Tan et al., 2006). Secondary antibodies were conjugated with AlexaFluor488 or AlexaFluor647 (Molecular Probes). DAPI (Sigma) was used to stain cell nuclei. Confocal images were acquired by using a Leica confocal microscope.

### Western Blot

HLFs, IPF donor cells, A549 cells lysates or lung tissue lysates were prepared in RIPA buffer. The patient lung biopsies study utilized biological specimens and data provided by the Lung Tissue Research Consortium (LTRC) supported by the National Heart, Lung, and Blood Institute (NHLBI). For Western blots, 1:1000-diluted mouse-anti-N-Cadherin (BD Biosciences), rabbit-anti-OB-Cadherin (Santa Cruz), mouse-anti-SMA (Sigma), mouse-anti-vimetin (DACO), mouse-anti-E-Cadherin (Abram), rabbit-anti-β-catenin (Cell Signaling Technology), anti-rabbit-p-Ser562-β-catenin (Cell Signaling Technology), rabbit-anti-β-actin (Santa Cruz) and mouse-anti-GAPDH (Santa Cruz) were used in combination with HRP-conjugated secondary antibodies (GE Healthcare). Western blots were developed using an ECL system (GE Healthcare). Quantification of the intensity of the bands was performed using NIH ImageJ software.

### Cell-Based Functional Assays

To assess functional responses, non-transfected HLFs or HLFs transfected with siRNAs were plated in full growth medium and left to grow overnight, starved for 24 h in serum-free medium and stimulated with different growth factors for a further 24 h. For FMT assessment, the HLF cells were stimulated with 5 ng/ml TGFβ (R&D Systems) or 10 μg/ml human N-Cadherin-Fc (R&D Systems), for collagen secretion—with 1 ng/ml TGFβ, for proliferation—with FBS, 10 ng/ml PDGF-BB (R&D Systems) or 10 ng/ml FGF-2 (R&D Systems) and for migration—with 10 μg/ml human N-Cadherin-Fc (R&D Systems) or human OB-Cadherin-Fc (R&D Systems). In the loss of function FMT experiment, 50 μg/ml N-Cadherin neutralizing antibody (clone GC-4, Sigma) was used. The level of FMT was evaluated by western blot for SMA. Collagen secretion was measured by Sircol collagen assay (Biocolor Life Science). Proliferation assay was performed using the DELFIA system (PerkinElmer), and migration was assessed with the Oris System (Platypus Technology).

For EMT assessment, A549 cells were simulated with 5 ng/ml TGFβ (R&D Systems), a combination of 5 ng/ml TGFβ (R&D Systems) and 2.5 ng/ml IL-1β (R&D Systems) or N-Cadherin-Fc (R&D Systems). The level of EMT was evaluated either by RT-PCR and Western blot for vimentin and E-Cadheirn or by immunocytochemistry for E-Cadherin. In the loss of function EMT experiment, N-Cadherin neutralizing tool antibody or IgG isotype control were used in the presence of 18 μg/ml N-cadheirn-Fc (R&D Systems).

### Statistical Analyses

Statistical significance was determined by using Student's t-test with two-tailed distribution and two-sample unequal variance. In all tests, two groups with only one changed parameter were compared.

### Example 1

### Cell-Cell Contacts Between Human Lung Fibroblasts Induce FMT

To investigate if cell-cell contacts between HLFs can induce FMT, we plated primary HLFs at low and at high cell density (LCD and HCD) and performed western blots to assess expression of the myofibroblast marker, SMA. Importantly, the amount of total protein was normalised prior to loading onto the gel. Experiments were performed in the presence and absence of TGFβ. As expected, TGFβ induced a marked increase in the expression of SMA in both, LCD and HCD. The presence of a small amount of SMA in non-stimulated cells has been reported previously and is due to some spontaneous expression in culture (Hinz et al., 2004). Interestingly, SMA expression was increased in cells plated at HCD, relative to cells plated at LCD, in both basal (non-stimulated) and TGFβ-stimulated conditions. These data suggest that cell-cell contacts may induce FMT.

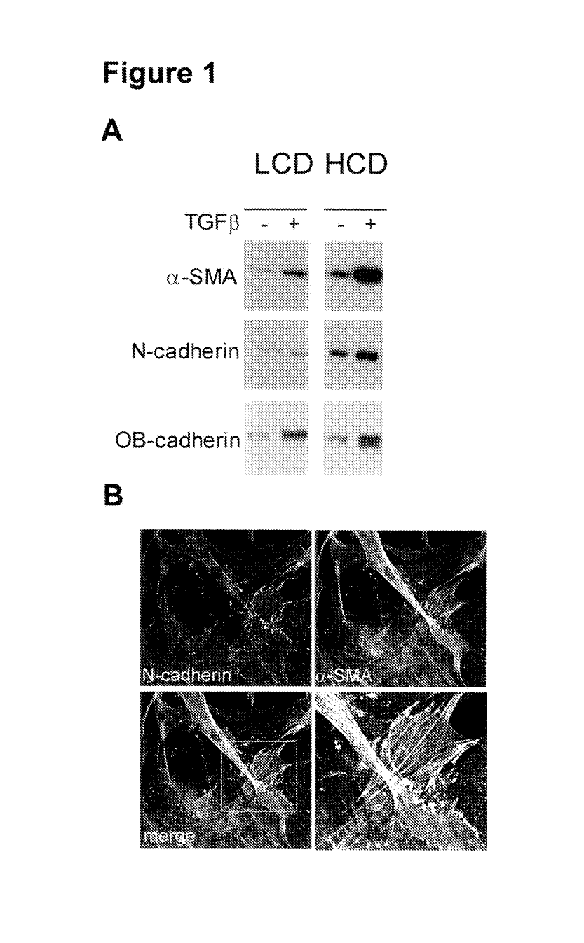

To further investigate the nature of the intercellular contacts that cause upregulation of SMA in HLFs, we investigated the expression levels in HLFs of the two main mesenchymal Cadherins at LCD and at HCD (FIG. 1A). N-Cadherin expression was considerably upregulated at HCD compared to LCD. OB-Cadherin expression was also increased at HCD though this was less pronounced than for N-Cadherin. TGFβ treatment upregulated the expression of N- and OB-Cadherin in both plating conditions. To further investigate a link between N-Cadherin and SMA expression, HLFs were stained for both SMA and N-Cadherin. Cells that form intercellular contacts tended to be SMA-positive and express N-Cadherin (FIG. 1B). These data demonstrated that when plated at high cell density HLFs form cell-cell junctions, express N-Cadherin and undergo FMT, thus representing the clinically relevant high cell-density fibroblast foci in IPF.

### Example 2

### N-Cadherin is Involved in Human Lung Fibroblast-to-Myofibroblast Transition in Vitro

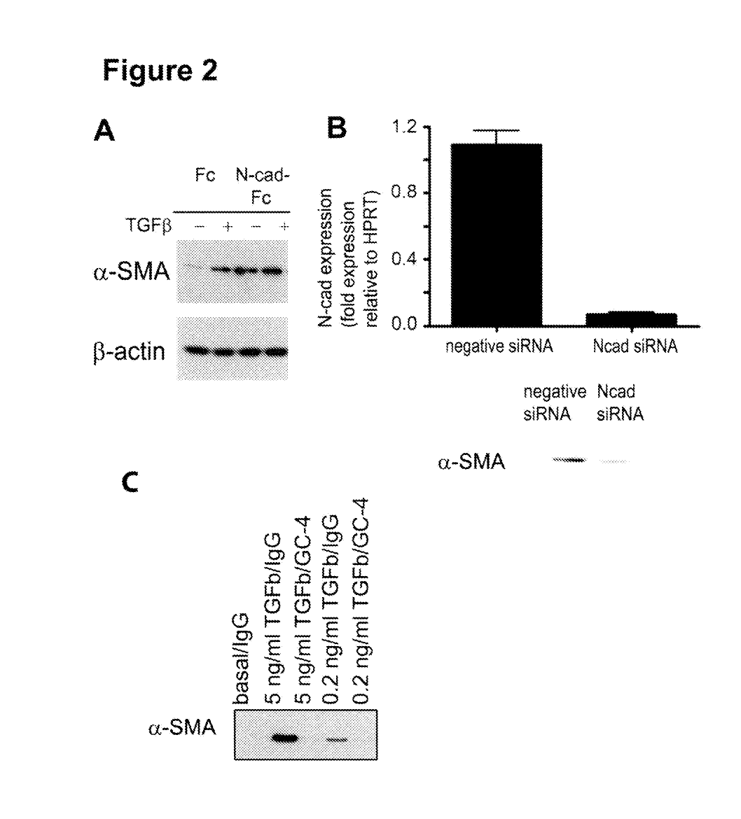

To further investigate the functional effects of N-Cadherin signalling in fibroblasts we performed gain- and loss-of function experiments using N-Cadherin-Fc fusion protein and siRNA, respectively. HLFs were treated with 10 μg/ml N-Cadherin-Fc or a control Fc-fragment in the presence and absence of TGFβ for 24 h, and SMA was measured by western blot (FIG. 2A). N-Cadherin-Fc has previously been demonstrated to mimic N-Cadherin homophilic interactions across cells and activate N-Cadherin signalling (Utton et al., 2001). N-Cadherin-Fc induced a significant increase in SMA expression to a similar level as seen with TGFβ. The control Fc-protein did not induce SMA expression. This result indicates that N-Cadherin activation can induce FMT to a similar extent as TGFβ. Interestingly, we did not observe an additive or synergistic effect with the combination of TGFβ and N-Cadherin-Fc on SMA expression in this time period.

In order to test whether N-Cadherin is a cause or an effect of FMT we transfected HLFs with siRNAs against N-Cadherin and checked the expression of SMA. HLFs transfected with siRNA to N-Cadherin had lower expression of N-Cadherin compared to control siRNA transfected cells (FIG. 2B). In addition, the knockdown of N-Cadherin resulted in a significant decrease of SMA expression in basal HLF cultures (FIG. 2B) demonstrating that N-Cadherin is a cause for FMT.

To test the hypothesis that interfering with N-Cadherin function rather than knockdown of protein expression affected FMT, we also incubated HLFs, in the presence or absence of TGFβ, with N-Cadherin blocking antibody (FIG. 2C) and measured SMA expression. N-Cadherin blocking antibody inhibited FMT to high and low dose TGFβ. These results confirmed that function blocking of surface N-Cadherin is sufficient to prevent TGFβ-induced SMA expression.

In conclusion, these experiments show that N-cadherin regulates FMT and inhibiting N-Cadherin function prevents this process.

### Example 3

### N-Cadherin Knockdown Inhibits Pro-Fibrotic Functional Effects in Human Lung Fibroblasts

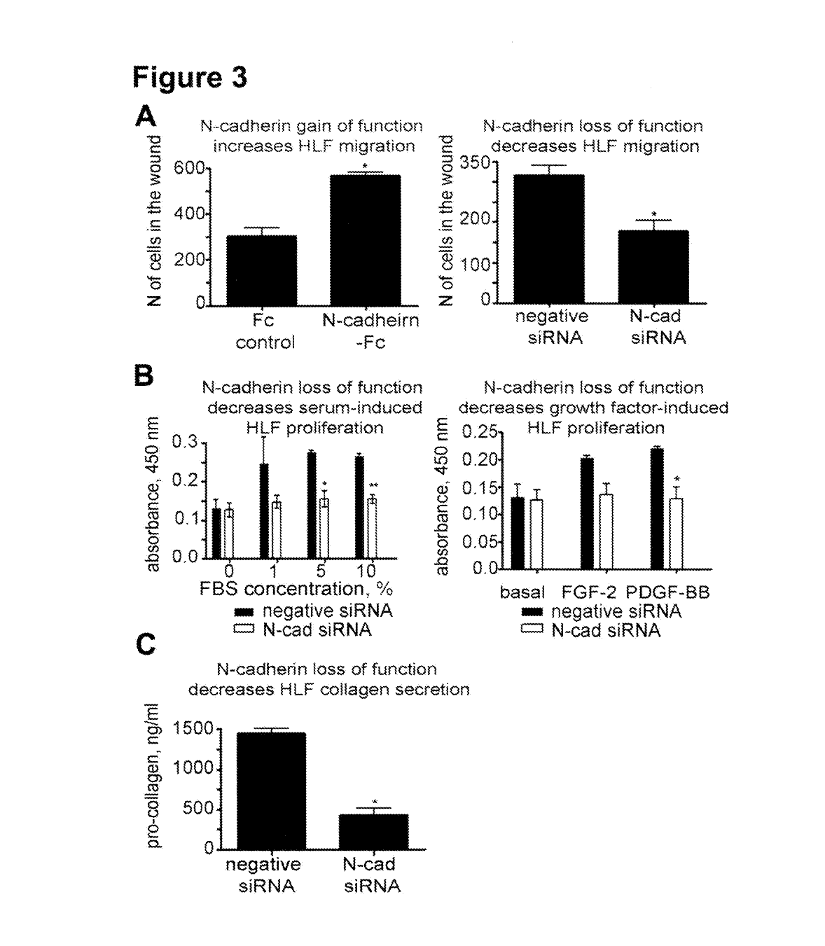

Increased migration is a hallmark of myofibroblasts and is thought to contribute to the pathology of IPF. Therefore, we investigated the effects of N-Cadherin gain- and loss-of function on the migratory potential of HLFs. We performed an in vitro wound closure assay with cells treated with the recombinant N-Cadherin-Fc chimera or with N-Cadherin siRNA. Activation of N-Cadherin signalling with N-Cadherin-Fc resulted in a significant increase in HLF migration into the wound. Knockdown of N-Cadherin with siRNA decreased migration into the wound (FIG. 3A).

Since N-Cadherin signalling has been shown to induce proliferation in some cells types (Mariotti et al., 2007) we assessed the possible effect of N-Cadherin on HLF proliferation. Cells were transfected with N-Cadherin siRNA and their proliferation examined in response to different concentrations of serum. N-Cadherin knockdown resulted in significantly decreased serum-induced proliferation (FIG. 3B). In order to study this effect in more detail we investigated the proliferative response of N-Cadherin siRNA transfected cells in response to FGF-2 and PDGF-BB (FIG. 3B). N-Cadherin knockdown resulted in a significant loss of proliferation in response to both factors. These results demonstrate that N-Cadherin signalling impacts HLF proliferation in response to a range of growth factors.

Since collagen deposition is a critical event in IPF we assessed the role of N-Cadherin in collagen secretion from HLFs. N-Cadherin siRNA transfected cells secrete significantly less collagen compared to cells transfected with control siRNA (FIG. 3C).

In conclusion, activation of N-Cadherin resulted in acquiring a myofibroblast phenotype, with cells displaying an increased migratory and proliferative response. N-Cadherin loss of function resulted in a marked decrease in all these effects and reduced collagen deposition, thus indicating that inhibiting N-Cadherin is likely to be beneficial in the treatment of IPF

### Example 4

### N-Cadherin Downstream Signalling in Human Lung Fibroblasts

N-Cadherin interacts directly with β-catenin, a downstream effector in the Wnt pathway, which either binds to α-catenin, directly linking the adherens junction to the cytoskeleton machinery, or translocates to the nucleus and acts as a transcription factor initiating the expression of various growth and differentiation genes. In addition, N-Cadherin signalling has demonstrated crosstalk with FGFR, c-Met, Wnt and PI3K-Akt in a cell type specific manner (De et al., 2004; Nelson and Nusse, 2004; Takeichi, 1991; Utton et al., 2001; Wallerand et al., 2010; Williams et al., 1994).

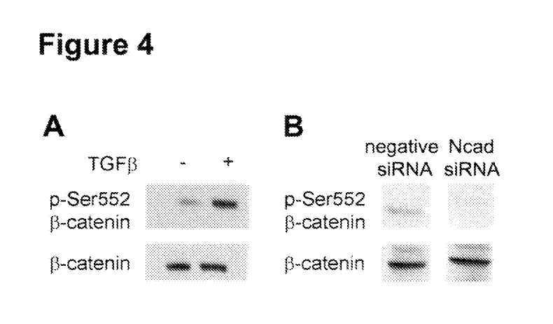

To assess the regulation of β-catenin in response to a fibrotic stimulus in HLFs, cells were stimulated with TGFβ and the expression levels and phosphorylation state of β-catenin were assessed by Western blot (FIG. 4A). There was no difference in the expression levels of total β-catenin in TGFβ-stimulated cells. However, when the cell lysates were probed with a phospho-specific β-catenin antibody, we found an increase in the phosphorylation of Ser552 upon TGFβ treatment. This phosphorylation site has recently been shown to be a substrate for AKT and activation of this pathway correlates with an increased cell survival rate (Fang et al., 2007; Horowitz et al., 2004). In order to investigate if β-catenin Ser552 phosphorylation in TGFβ-stimulated HLFs is downstream of N-Cadherin, HLFs were transfected with N-Cadherin siRNA and probed using the phospho-Ser552 specific antibody (FIG. 4B). Knockdown of N-Cadherin resulted in almost complete inhibition of the Ser552 phosphorylation on β-catenin without affecting the total β-catenin levels in the cells. These results indicate that N-Cadherin is involved in TGFβ-induced pro-survival signals in myofibroblasts through crosstalk with the PI3K-AKT pathway. As both, Wnt and PI3K-AKT pathways are upregulated in IPF, These results underline the therapeutic potential of N-Cadherin in IPF.

### Example 5

### N- to OB-Cadherin Switch in Human Lung Fibroblasts

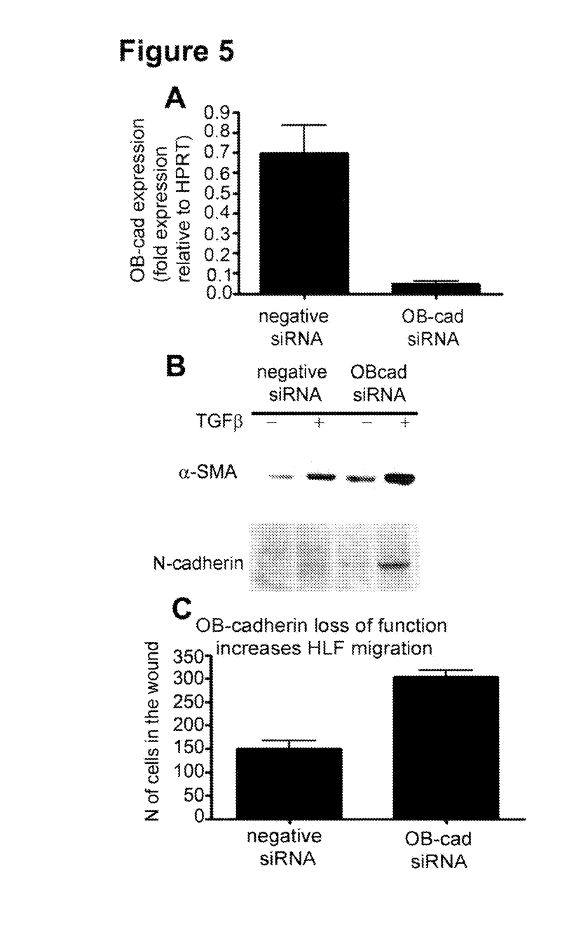

OB-Cadherin is a non-classical Cadherin highly expressed in osteoblasts (Boscher and Mege, 2008). OB-Cadherin forms strong interactions and is associated with stable adhesion and activation of proliferative signalling pathways (Boscher and Mege, 2008; Cavallaro et al., 2002; Gumbiner, 2005b; Nelson and Nusse, 2004; Patel et al., 2003). Since OB-Cadherin is expressed in cells of the mesenchyme lineage we investigated expression of this protein in basal and stimulated HLFs. OB-Cadherin expression was detected at low levels in HLFs growing in culture. Interestingly, OB-Cadherin was upregulated in response to TGFβ. This upregulation of OB-Cadherin seems to occur concomitantly with upregulation of SMA and N-Cadherin expression (FIG. 1A). To dissect the order of events further and to investigate the role of OB-Cadherin in FMT, HLFs were transfected with either control or OB-Cadherin siRNA and expression of SMA was measured. Cells transfected with OB-cadheren siRNA expressed reduced levels of OB-Cadherin compared to control cells (FIG. 5A). Surprisingly however, OB-Cadherin siRNA transfected cells expressed more SMA. Since this was in marked contrast to our observations with N-Cadherin we also assessed the effect of OB-Cadherin knockdown on N-Cadherin levels. OB-Cadherin siRNA transfected cells expressed significantly increased levels of N-Cadherin compared to control cells (FIG. 5B). These results demonstrate that N-Cadherin expression correlates with SMA expression in TGFβ stimulated HLFs and that, whilst OB-Cadherin expression correlates with SMA expression in control HLFs, in cells transfected with OB-cadherin siRNA there is an increase in SMA expression due to a compensatory N-Cadherin upregulation. Together these results suggest that fibroblasts stimulated with TGFβ sequentially express N- and then OB-Cadherin and that knockdown of OB-Cadherin traps cells in the N-Cadherin expressing stage. This then leads to an increase in SMA expression. Since one of the functional effects of N-Cadherin signalling was to increase cell migration we tested the effect of OB-Cadherin siRNA on this response. Consistent with the results above, OB-Cadherin siRNA transfected cells exhibited an increase in migration (FIG. 5C).

Therefore, inhibiting N-Cadherin and not OB-Cadherin in IPF is more likely to provide therapeutic benefit.

### Example 6

### N-Cadherin Expression During EMT

There is growing evidence that during IPF progression, epithelial cells undergo EMT (Horowitz and Thannickal, 2006; Kim et al., 2006; Nieman et al., 1999; Selman et al., 2008). During EMT, epithelial cells lose expression of E-Cadherin and a Cadherin switch towards de novo expressed N-Cadherin occurs. The loss of the stable cell-cell contacts mediated by E-Cadherin and the gain of the more dynamic N-Cadherin junctions provide the signal for these cells to detach from the epithelial sheet and migrate. The gain of this mesenchymal phenotype is characterised by the loss of epithelial markers including E-Cadherin and gain of mesenchymal markers including vimentin (Cavallaro et al., 2002; Shintani et al., 2008; Thiery et al., 2009).

In vitro this process can be mimicked by treating epithelial cells with a combination of TGFβ and IL-1β (Border and Noble, 1994; Leivonen et al., 2002; Massague, 2008; Willis et al., 2005).

We investigated the expression and the role of N-Cadherin during EMT using A549 cells, a cell line derived from human alveolar type II epithelial cells, or primary human bronchial epithelial cells (HBECs).

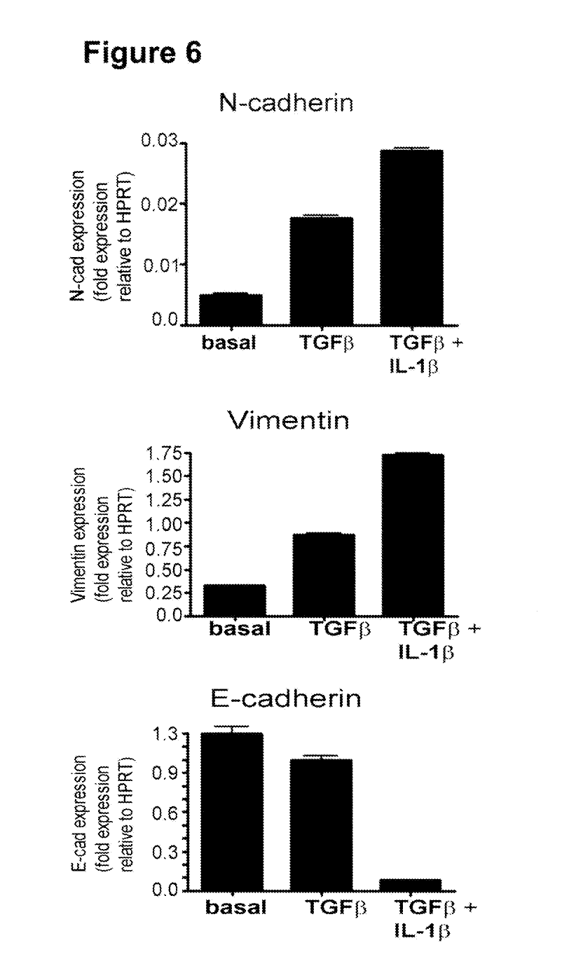

A549 cells were treated with TGFβ in combination with IL-1β for 48 hours in order to induce EMT. Consistent with previous reports (Kim et al., 2006; Willis et al., 2005) this induced a loss of E-Cadherin, gain of vimentin expression and cell elongation. During this process expression levels of these EMT markers and N-Cadherin were monitored using real-time PCR (FIG. 6). Treatment of A549 cells with TGFβ and IL-1β resulted in a 5 fold increase in N-Cadherin mRNA expression, thus confirming that N-Cadherin can be used as a marker for EMT (Kim et al., 2006; Willis et al., 2005)

### Example 7

### N-Cadherin Loss of Function Prevents EMT

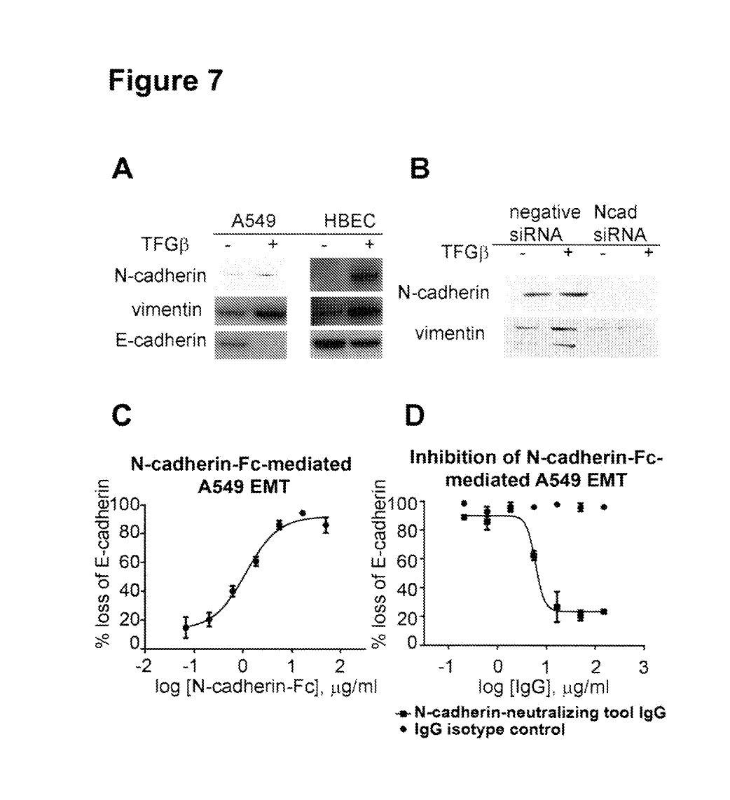

The increased N-cadherin expression in cells treated with TGFβ in combination with IL-1β was confirmed as an increase in N-Cadherin protein (FIG. 7A). It is important to note that we detected basal N-Cadherin and vimentin expression in non-treated A549 cells, which is most likely due to the fact that this is a transformed cell line. In order to confirm our observation in primary epithelial cells, which do not express N-Cadherin when untreated, we used primary HBECs. We performed western blot analysis with lysates from non-treated and TGFβ plus IL-1β treated cells and detected the de novo expression of N-Cadherin (FIG. 7A). To investigate the effect of N-Cadherin loss of function on EMT, we transfected A549 cells with N-Cadherin siRNA or control siRNA and assessed the EMT (FIG. 7B). TGFβ and IL-1β treatment of control siRNA transfected cells induced the EMT specific changes in morphology, i.e. cell elongation and a loss of the characteristic strong epithelial adhesion in groups of cells. By using Western blot we could also detect an increase in vimentin expression. However, in the N-Cadherin knockdown cells epithelial morphology was partially preserved. Moreover, we found that N-Cadherin knockdown completely prevented the upregulation of vimentin expression upon TGFβ and IL-1β treatment. These results demonstrate that N-Cadherin regulates key cellular processes guiding EMT.

To confirm that N-Cadherin gain and loss of function rather than siRNA-mediated knockdown regulate EMT, we have treated A549 cells with recombinant N-Cadherin-Fc in the presence or absence of a tool N-Cadheirn neutralizing antibody (FIG. 7C). We measured EMT by using immunocytochemistry for E-Cadherin. In the gain of function setup of the experiment, addition of recombinant N-Cadherin-Fc caused significant EMT, measured by decreased E-Cadherin signal. The extent of EMT was comparable to the one caused by the control treatment with TGFβ and IL-1β. In the loss of function setup, the cells were pre-incubated with neutralizing antibody before treatment with N-Cadherin-Fc. This resulted in complete inhibition of EMT, thus confirming that inhibition of N-Cadherin result in prevention of this process in IPF.

### Example 8

### N-Cadherin Expression in Animal Models of Pulmonary Fibrosis and in IPF Patient-Derived Lung Fibroblasts

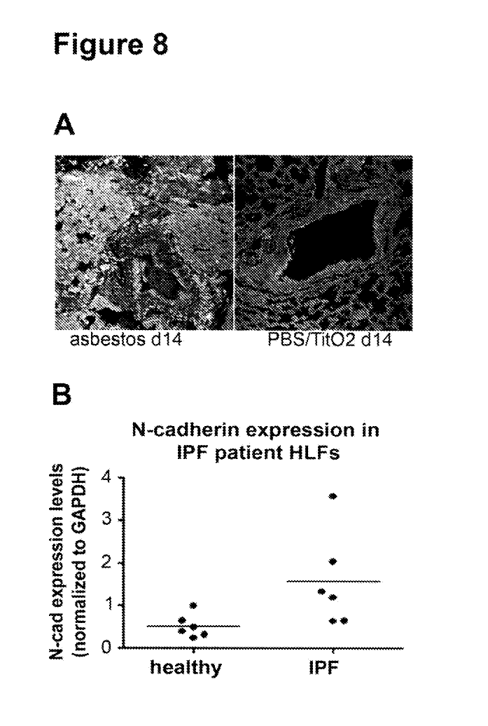

To assess a possible role for N-Cadherin in IPF we analysed the expression of N-Cadherin in a pre-clinical animal model of pulmonary fibrosis—the asbestos mouse model (Tan et al., 2006). Asbestos is a naturally occurring silicate mineral, which if inhaled causes progressive lung fibrosis. We treated mice with asbestos, a control mineral—titanium dioxide (TitO₂) or saline solution over a period of 64 days, animals were sacrificed at various time points, lungs harvested and separate samples processed for IHC analysis. Immunostaining for SMA in sections from lung tissue in the three groups is usually observed around the blood vessels in the vascular smooth muscle cells. Importantly however, in the asbestos treated group there is consistently an increase of the SMA signal in the surrounding fibrotic tissue. Immunostaining for N-Cadherin demonstrated that regions undergoing remodelling are the ones expressing N-Cadherin and that N-cadherin is not expressed in control animals (FIG. 8A) These data demonstrate that N-Cadherin is upregulated in the fibrotic regions of the lung in a pre-clinical mouse model of IPF.

To look for a disease relevant cell type specific expression of N-Cadherin we analysed N-Cadherin expression in fibroblasts purified from the lungs of IPF patients by western blot. Human lung fibroblasts from donors without IPF were used as a control. There was a high level of variation in N-Cadherin expression in IPF cells. However, the patient cells expressed significantly more N-Cadherin than control cells (FIG. 8B).

### Example 9

### N-Cadherin Expression in IPF Patients Lung Biopsies

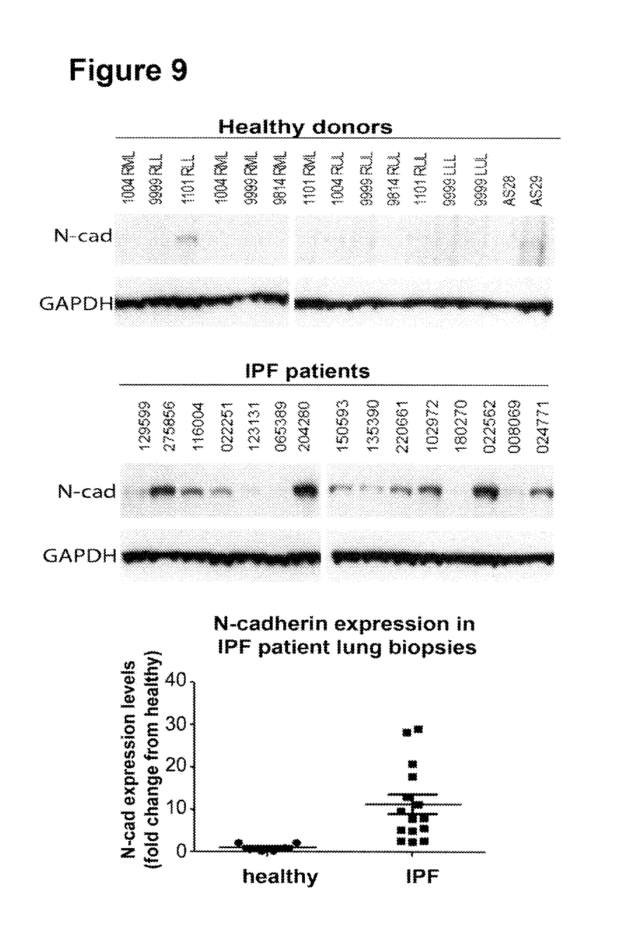

In addition, we have analysed the expression of N-Cadherin in IPF patient lung biopsies compared to healthy controls by Western blot (FIG. 9). We found that N-cadherin is significantly upregulated in most of the analysed IPF patients lung tissues compared to the healthy controls, thus providing additional clinical support for a role of N-cadheirn in IPF.

### DISCUSSION

Physiological wound healing is a defensive mechanism occurring naturally in the organism in response to injury. The distinct stages of the wound healing process are tightly regulated by direct and indirect cellular communication. It is widely accepted that FMT is a key event in wound healing, necessary to provide the mechanical basis for restoration of the tissue architecture. Similarly to other fibrotic indications, such as scleroderma, kidney and liver fibrosis, IPF is believed to represent an abnormal wound healing response as a result of repeated micro-injury and failure of resolution.

TGFβ is known as a fibrogenic master switch in various fibrotic conditions synergising with other cytokines to elicit effects on the development and progression of IPF (du Bois, 2010). An increasing number of studies demonstrate that aspects of IPF progression are regulated by cell-extracellular matrix interactions and direct cell-cell interactions (Hinz and Gabbiani, 2003a). For example, applying mechanical stress on fibroblasts leads to induction of SMA expression, while removing mechanical stress reduces SMA expression (Follonier et al., 2008; Hinz et al., 2001; Hinz and Gabbiani, 2003b; Hinz and Gabbiani, 2003a). The mechanical characteristic of the matrix appears to modulate TGFβ action in order to promote either migration or contraction (Wipff et al., 2007). Furthermore this is likely to form a positive feedback loop as the tissue becomes more rigid increasing latent TGFβ activation in response to cell contractility in the non-compliant micro-environment.

It has been suggested previously that upon injury in the lung, the integrity of the basement membrane separating stromal cells from the epithelium is compromised thus allowing for a direct cell-cell contact between fibroblasts and epithelial cells. Interestingly, these direct contacts were demonstrated to induce FMT and to promote survival in the fibroblasts, and at the same time to induce apoptosis in the epithelial cells (Horowitz and Thannickal, 2006), suggesting that direct interference with cell-cell adhesion might be a viable therapeutic strategy for the treatment of IPF. Consistent with this idea the data presented here demonstrates a fundamental role for cell-cell contacts and N-Cadherin in driving fibrotic cellular responses. Furthermore, N-Cadherin is upregularted in IPF patient lung biopsies, the asbestos pre-clinical model of IPF and in lung fibroblasts from IPF patients.

A recently proposed aspect of IPF development and progression is EMT supplying an additional source of myofibroblasts from the damaged epithelium (Selman et al., 2006; Selman et al., 2008). Fully differentiated epithelial cells undergo a transition into mesenchymal cells. The Cadherin switch from E- to N-Cadherin provides cells undergoing EMT with a migratory potential and might regulate further FMT, proliferation and survival rates. We have demonstrated that N-Cadherin inhibition prevents EMT in alveolar type II epithelial cell line. Therefore, inhibiting the activity of N-Cadherin in vivo is likely to prevent or reduce EMT, and limit one of the sources of myofibroblasts accumulating in the IPF lung.

Myofibroblasts represent the core of all fibrotic diseases. In the present study, we demonstrate that N-Cadherin is expressed predominantly by myofibroblasts in contrast to normal fibroblasts. This observation agrees with previous reports on junction formation in fibroblast cultures (Hinz et al., 2004). Moreover, we provide evidence for the function of N-Cadherin in myofibroblasts. Similar to epithelial cells undergoing EMT, fibroblasts undergoing FMT express de novo N-Cadherin, and this provides them with a pro-migratory, pro-proliferative and pro-survival phenotype. This is confirmed in the present study since interfering with N-Cadherin function results in downregulation of migration, proliferation and survival. The formation of the myofibroblast itself depends on N-Cadherin signalling since activation of N-Cadherin induced SMA expression and knockdown of N-Cadherin inhibited FMT. Importantly, interfering with N-Cadherin function by using a function blocking antibody in our study resulted in downregulation of TGFβ-induced FMT. This observation indicates that a blocking antibody approach for inhibition of N-Cadherin is likely tot prove therapeutically valid for IPF treatment.

N-Cadherin also regulates abnormal collagen secretion by activated myofibroblasts a critical feature of IPF pathology. In summary, N-Cadherin is involved in many of the myofibroblast functions that contribute to IPF as well as the formation of myofibroblasts by FMT and EMT (Nieman et al., 1999). Therefore, an agent blocking N-Cadherin function could inhibit pro-fibrotic functions of the fibroblasts/myofibroblasts driven by cell-cell junctions formation and lead to a slowing or arrest of disease progression (Hinz, 2004). N-Cadherin also couples to a pro-survival signalling pathway and N-Cadherin blockade may lead to clearance of fibrotic lesions and reversal of lung fibrosis.

N-Cadherin is already a drug target for multiple oncology indications, where it promotes angiogenesis and tumour cells migration, proliferation and survival (Augustine et al., 2008; Mariotti et al., 2007). A cyclic peptide N-Cadherin antagonist, Exherin or ADH-1, has been tested clinically and is currently in phase 2 trials in cancer and a recent study suggested the use of N-Cadherin antibodies in diagnosing, evaluating and treating N-Cadherin-related cancer and described the use of such antibodies in treating pre-clinical models of prostate cancer (Tanaka et al., 2010). These data demonstrate that interfering with N-Cadherin function with an antibody or low molecular weight molecule is a viable therapeutic approach. Interestingly, the N-Cadherin antagonist, ADH-1, has been shown to affect tumour blood flow by disrupting the neo-vasculature around malignant melanoma, without affecting normal blood flow in surrounding tissue. The integrity of these blood vessels are maintained by N-Cadherin mediated cellular junctions before the more stable VE-Cadherin mediated junctions take over. In IPF the areas around the fibrotic foci are highly vascularised with malformed vessels similar to those in the tumour microenvironment and so it is tempting to speculate that N-Cadherin blockade may impact these areas of the IPF lung as well as the densely packed fibrotic foci.

In conclusion, we have looked for expression and functional role for N-Cadherin in disease relevant fibroblasts cells or tissues from normal and IPF patients. We found that N-Cadherin was upregulted in the fibrotic regions and in the main cell type responsible for the IPF phenotype—the myofibroblasts. N-Cadherin loss of function resulted in inhibition of all processes associated with this fibrotic phenotype—FMT, proliferation, collagen secretion, migration and EMT. Therefore, we conclude that inhibition of N-Cadherin in IPF patients is very likely to provide a therapeutic benefit.

### REFERENCES

### Reference List

Abe, R., Donnelly, S. C., Peng, T., Bucala, R., and Metz, C. N. (2001). Peripheral blood fibrocytes: differentiation pathway and migration to wound sites. J. Immunol. 166, 7556-7562.
American Thoracic Society, E. R. S. (2000). Idiopathic Pulmonary Fibrosis: Diagnosis and Treatment. International Consensus Statement. Am. J. Respir. Crit. Care Med. 161, 646-664.
Augustine, C. K., Yoshimoto, Y., Gupta, M., Zipfel, P. A., Selim, M. A., Febbo, P., Pendergast, A. M., Peters, W. P., and Tyler, D. S. (2008). Targeting N-Cadherin enhances antitumor activity of cytotoxic therapies in melanoma treatment. Cancer Res. 68, 3777-3784.
B.T.SOCIETY and S.O.COMMITTEE. The Diagnosis, Assessment and Treatment of Diffuse Parenchymal Lung Disease in Adults. Thorax 54(Suppl 1)[April], S1-S-28. 1999. Ref Type: Journal (Full)
Barbarin, V., Nihoul, A., Misson, P., Arras, M., Delos, M., Leclercq, I., Lison, D., and Huaux, F. (2005). The role of pro- and anti-inflammatory responses in silica-induced lung fibrosis
2. Respir. Res. 6, 112.
Blaschuk, O. W. and Rowlands, T. M. (2000). Cadherins as modulators of angiogenesis and the structural integrity of blood vessels
2. Cancer Metastasis Rev. 19, 1-5.
Border, W. A. and Noble, N. A. (1994). Transforming growth factor beta in tissue fibrosis
1. N. Engl. J. Med. 331, 1286-1292.
Boscher, C. and Mege, R. M. (2008). Cadherin-11 interacts with the FGF receptor and induces neurite outgrowth through associated downstream signalling
2. Cell Signal. 20, 1061-1072.
Bouros, Demosthenes. Pirfenidone for idiopathic pulmonary fibrosis. The Lancet 377[9779], 1727-1729. 21-5-2011.
Ref Type: Abstract
Burden-Gulley, S. M., Gates, T. J., Craig, S. E., Lou, S. F., Oblander, S. A., Howell, S., Gupta, M., and Brady-Kalnay, S. M. (2009). Novel peptide mimetic small molecules of the HAV motif in N-cadherin inhibit N-cadherin-mediated neurite outgrowth and cell adhesion. Peptides 30, 2380-2387.
Cavallaro, U., Schaffhauser, B., and Christofori, G. (2002). Cadherins and the tumour progression: is it all in a switch?
1. Cancer Lett. 176, 123-128.
Chatziantoniou, C. and Dussaule, J. C. (2005). Insights into the mechanisms of renal fibrosis: is it possible to achieve regression? American Journal of Physiology—Renal Physiology 289, F227-F234.
Davis, G. S., Leslie, K. O., and Hemenway, D. R. (1998a). Silicosis in mice: effects of dose, time, and genetic strain
3. J. Environ. Pathol. Toxicol. Oncol. 17, 81-97.
Davis, G. S., Pfeiffer, L. M., and Hemenway, D. R. (1998b). Persistent overexpression of interleukin-1 beta and tumor necrosis factor-alpha in murine silicosis
2. J. Environ. Pathol. Toxicol. Oncol. 17, 99-114.
De, W. O., Westbroek, W., Verloes, A., Bloemen, N., Bracke, M., Gespach, C., Bruyneel, E., and Mareel, M. (2004). Critical role of N-Cadherin in myofibroblast invasion and migration in vitro stimulated by colon-cancer-cell-derived TGF-beta or wounding. J. Cell Sci. 117, 4691-4703.
Douglas, W. W., Ryu, J. H., and Schroeder, D. R. (2000). Idiopathic pulmonary fibrosis: Impact of oxygen and colchicine, prednisone, or no therapy on survival
2. Am. J. Respir. Crit Care Med. 161, 1172-1178.
du Bois, R. M. (2010). Strategies for treating idiopathic pulmonary fibrosis. Nat. Rev. Drug Discov. 9, 129-140.
Engelhardt, J. F., Litzky, L., and Wilson, J. M. (1994). Prolonged transgene expression in cotton rat lung with recombinant adenoviruses defective in E2a
9. Hum. Gene Ther. 5, 1217-1229.
Fang, D., Hawke, D., Zheng, Y., Xia, Y., Meisenhelder, J., Nika, H., Mills, G. B., Kobayashi, R., Hunter, T., and Lu, Z. (2007). Phosphorylation of beta-catenin by AKT promotes beta-catenin transcriptional activity. J. Biol. Chem. 282, 11221-11229.
Farina, A. K., Bong, Y. S., Feltes, C. M., and Byers, S. W. (2009). Post-transcriptional regulation of cadherin-11 expression by GSK-3 and beta-catenin in prostate and breast cancer cells
1. PLoS. One. 4, e4797.
Follonier, L., Schaub, S., Meister, J. J., and Hinz, B. (2008). Myofibroblast communication is controlled by intercellular mechanical coupling. J. Cell Sci. 121, 3305-3316.
Friedman, S. L. (2004). Mechanisms of disease: Mechanisms of hepatic fibrosis and therapeutic implications
2. Nat. Clin. Pract. Gastroenterol. Hepatol. 1, 98-105.
Gharaee-Kermani, M. and Phan, S. H. (2005). Molecular mechanisms of and possible treatment strategies for idiopathic pulmonary fibrosis. Curr. Pharm. Des 11, 3943-3971.
Gumbiner, B. M. (2005a). Coordinate gene regulation by two different catenins
1. Dev. Cell 8, 795-796.
Gumbiner, B. M. (2005b). Regulation of cadherin-mediated adhesion in morphogenesis
1. Nat. Rev. Mol. Cell Biol. 6, 622-634.
Hinz, B. (2007). Formation and function of the myofibroblast during tissue repair
2. J. Invest Dermatol. 127, 526-537.
Hinz, B., Dugina, V., Ballestrem, C., Wehrle-Haller, B., and Chaponnier, C. (2003). Alpha-smooth muscle actin is crucial for focal adhesion maturation in myofibroblasts
2. Mol. Biol. Cell 14, 2508-2519.
Hinz, B. and Gabbiani, G. (2003a). Cell-matrix and cell-cell contacts of myofibroblasts: role in connective tissue remodeling. Thromb. Haemost. 90, 993-1002.
Hinz, B. and Gabbiani, G. (2003b). Mechanisms of force generation and transmission by myofibroblasts. Curr. Opin. Biotechnol. 14, 538-546.
Hinz, B., Mastrangelo, D., Iselin, C. E., Chaponnier, C., and Gabbiani, G. (2001). Mechanical tension controls granulation tissue contractile activity and myofibroblast differentiation. Am. J. Pathol. 159, 1009-1020.
Hinz, B., Phan, S. H., Thannickal, V. J., Galli, A., Bochaton-Piallat, M. L., and Gabbiani, G. (2007). The myofibroblast: one function, multiple origins. Am. J. Pathol. 170, 1807-1816.
Hinz, B., Pittet, P., Smith-Clerc, J., Chaponnier, C., and Meister, J. J. (2004). Myofibroblast development is characterized by specific cell-cell adherens junctions. Mol. Biol. Cell 15, 4310-4320.
Horowitz, J. C., Cui, Z., Moore, T. A., Meier, T. R., Reddy, R. C., Toews, G. B., Standiford, T. J., and Thannickal, V. J. (2006). Constitutive activation of prosurvival signaling in alveolar mesenchymal cells isolated from patients with nonresolving acute respiratory distress syndrome
2. Am. J. Physiol Lung Cell Mol. Physiol 290, L415-L425.
Horowitz, J. C., Lee, D. Y., Waghray, M., Keshamouni, V. G., Thomas, P. E., Zhang, H., Cui, Z., and Thannickal, V. J. (2004). Activation of the pro-survival phosphatidylinositol 3-kinase/AKT pathway by transforming growth factor-beta1 in mesenchymal cells is mediated by p38 MAPK-dependent induction of an autocrine growth factor. J. Biol. Chem. 279, 1359-1367.
Horowitz, J. C. and Thannickal, V. J. (2006). Epithelial-mesenchymal interactions in pulmonary fibrosis. Semin. Respir. Crit Care Med. 27, 600-612.
Kaneko, M., Inoue, H., Nakazawa, R., Azuma, N., Suzuki, M., Yamauchi, S., Margolin, S. B., Tsubota, K., and Saito, I. (1998). Pirfenidone induces intercellular adhesion molecule-1 (ICAM-1) down-regulation on cultured human synovial fibroblasts
2. Clin. Exp. Immunol. 113, 72-76.
Kim, K. K., Kugler, M. C., Wolters, P. J., Robillard, L., Galvez, M. G., Brumwell, A. N., Sheppard, D., and Chapman, H. A. (2006). Alveolar epithelial cell mesenchymal transition develops in vivo during pulmonary fibrosis and is regulated by the extracellular matrix
3. Proc. Natl. Acad. Sci. U.S.A 103, 13180-13185.
King, T. E., Jr., Pardo, A., and Selman, M. (2011). Idiopathic pulmonary fibrosis
1. Lancet 378, 1949-1961.
Leivonen, S. K., Chantry, A., Hakkinen, L., Han, J., and Kahari, V. M. (2002). Smad3 mediates transforming growth factor-beta-induced collagenase-3 (matrix metalloproteinase-13) expression in human gingival fibroblasts. Evidence for cross-talk between Smad3 and p38 signaling pathways
1. J. Biol. Chem. 277, 46338-46346.
Luo, Y. and Radice, G. L. (2005). N-Cadherin acts upstream of VE-cadherin in controlling vascular morphogenesis
2. J. Cell Biol. 169, 29-34.
Mapel, D. W., Samet, J. M., and Coultas, D. B. (1996). Corticosteroids and the treatment of idiopathic pulmonary fibrosis. Past, present, and future
6. Chest 110, 1058-1067.
Mariotti, A., Perotti, A., Sessa, C., and Ruegg, C. (2007). N-Cadherin as a therapeutic target in cancer. Expert. Opin. Investig. Drugs 16, 451-465.
Massague, J. (2008). TGFbeta in Cancer
2. Cell 134, 215-230.
Meltzer, E. B. and Noble, P. W. (2008). Idiopathic pulmonary fibrosis
3. Orphanet. J. Rare. Dis. 3, 8.
Moeller, A., Ask, K., Warburton, D., Gauldie, J., and Kolb, M. (2008). The bleomycin animal model: a useful tool to investigate treatment options for idiopathic pulmonary fibrosis?
3. Int. J. Biochem. Cell Biol. 40, 362-382.
Moore, B. B. and Hogaboam, C. M. (2008). Murine models of pulmonary fibrosis
4. Am. J. Physiol Lung Cell Mol. Physiol 294, L152-L160.
Muggia, F. M., Louie, A. C., and Sikic, B. I. (1983). Pulmonary toxicity of antitumor agents
3. Cancer Treat. Rev. 10, 221-243.
Nelson, W. J. and Nusse, R. (2004). Convergence of Wnt, beta-catenin, and cadherin pathways
2. Science 303, 1483-1487.
Nieman, M. T., Prudoff, R. S., Johnson, K. R., and Wheelock, M. J. (1999). N-Cadherin promotes motility in human breast cancer cells regardless of their E-cadherin expression. J. Cell Biol. 147, 631-644.
Patel, S. D., Chen, C. P., Bahna, F., Honig, B., and Shapiro, L. (2003). Cadherin-mediated cell-cell adhesion: sticking together as a family
2. Curr. Opin. Struct. Biol. 13, 690-698.
Perel, P., Roberts, I., Sena, E., Wheble, P., Briscoe, C., Sandercock, P., Macleod, M., Mignini, L. E., Jayaram, P., and Khan, K. S. (2007). Comparison of treatment effects between animal experiments and clinical trials: systematic review
28. BMJ 334, 197.
Phan, S. H. (2002). The myofibroblast in pulmonary fibrosis
1. Chest 122, 286S-289S.
Phan, S. H., Armstrong, G., Sulavik, M. C., Schrier, D., Johnson, K. J., and Ward, P. A. (1983). A comparative study of pulmonary fibrosis induced by bleomycin and an 02 metabolite producing enzyme system
3. Chest 83, 44S-45S.
Phan, S. H., Thrall, R. S., and Ward, P. A. (1980). Bleomycin-induced pulmonary fibrosis in rats: biochemical demonstration of increased rate of collagen synthesis
5. Am. Rev. Respir. Dis. 121, 501-506.
Phillips, R. J., Burdick, M. D., Hong, K., Lutz, M. A., Murray, L. A., Xue, Y. Y., Belperio, J. A., Keane, M. P., and Strieter, R. M. (2004). Circulating fibrocytes traffic to the lungs in response to CXCL12 and mediate fibrosis
4. J. Clin. Invest 114, 438-446.
Raghu, G., Chen, Y. Y., Rusch, V., and Rabinovitch, P. S. (1988). Differential proliferation of fibroblasts cultured from normal and fibrotic human lungs
1. Am. Rev. Respir. Dis. 138, 703-708.
Raghu, G., Collard, H. R., Egan, J. J., Martinez, F. J., Behr, J., Brown, K. K., Colby, T. V., Cordier, J. F., Flaherty, K. R., Lasky, J. A., Lynch, D. A., Ryu, J. H., Swigris, J. J., Wells, A. U., Ancochea, J., Bouros, D., Carvalho, C., Costabel, U., Ebina, M., Hansell, D. M., Johkoh, T., Kim, D. S., King, T. E., Jr., Kondoh, Y., Myers, J., Muller, N. L., Nicholson, A. G., Richeldi, L., Selman, M., Dudden, R. F., Griss, B. S., Protzko, S. L., and Schunemann, H. J. (2011). An official ATS/ERS/JRS/ALAT statement: idiopathic pulmonary fibrosis: evidence-based guidelines for diagnosis and management
1. Am. J. Respir. Crit Care Med. 183, 788-824.
Raghu, G., Mageto, Y. N., Lockhart, D., Schmidt, R. A., Wood, D. E., and Godwin, J. D. (1999). The accuracy of the clinical diagnosis of new-onset idiopathic pulmonary fibrosis and other interstitial lung disease: A prospective study
9. Chest 116, 1168-1174.
Roberts, S. N., Howie, S. E., Wallace, W. A., Brown, D. M., Lamb, D., Ramage, E. A., and Donaldson, K. (1995). A novel model for human interstitial lung disease: hapten-driven lung fibrosis in rodents. J. Pathol. 176, 309-318.
Schrier, D. J., Kunkel, R. G., and Phan, S. H. (1983). The role of strain variation in murine bleomycin-induced pulmonary fibrosis
2. Am. Rev. Respir. Dis. 127, 63-66.
Scotton, C. J. and Chambers, R. C. (2007). Molecular targets in pulmonary fibrosis: the myofibroblast in focus. Chest 132, 1311-1321.
Sebe, A., Leivonen, S. K., Fintha, A., Masszi, A., Rosivall, L., Kahari, V. M., and Mucsi, I. (2008). Transforming growth factor-beta-induced alpha-smooth muscle cell actin expression in renal proximal tubular cells is regulated by p38beta mitogen-activated protein kinase, extracellular signal-regulated protein kinase 1,2 and the Smad signalling during epithelial-myofibroblast transdifferentiation
1. Nephrol. Dial. Transplant. 23, 1537-1545.
Selman, M., Carrillo, G., Salas, J., Padilla, R. P., Perez-Chavira, R., Sansores, R., and Chapela, R. (1998). Colchicine, D-penicillamine, and prednisone in the treatment of idiopathic pulmonary fibrosis: a controlled clinical trial
13. Chest 114, 507-512.
Selman, M., King, T. E., and Pardo, A. (2001). Idiopathic pulmonary fibrosis: prevailing and evolving hypotheses about its pathogenesis and implications for therapy
1. Ann. Intern. Med. 134, 136-151.
Selman, M., Pardo, A., Barrera, L., Estrada, A., Watson, S. R., Wilson, K., Aziz, N., Kaminski, N., and Zlotnik, A. (2006). Gene expression profiles distinguish idiopathic pulmonary fibrosis from hypersensitivity pneumonitis. Am. J. Respir. Crit Care Med. 173, 188-198.
Selman, M., Pardo, A., and Kaminski, N. (2008). Idiopathic pulmonary fibrosis: aberrant recapitulation of developmental programs? PLoS. Med. 5, e62.
Sharplin, J. and Franko, A. J. (1989). A quantitative histological study of strain-dependent differences in the effects of irradiation on mouse lung during the early phase
11. Radiat. Res. 119, 1-14.
Shintani, Y., Maeda, M., Chaika, N., Johnson, K. R., and Wheelock, M. J. (2008). Collagen I promotes epithelial-to-mesenchymal transition in lung cancer cells via transforming growth factor-beta signaling
2. Am. J. Respir. Cell Mol. Biol. 38, 95-104.
Takeichi, M. (1991). Cadherin cell adhesion receptors as a morphogenetic regulator. Science 251, 1451-1455.
Tan, R. J., Fattman, C. L., Niehouse, L. M., Tobolewski, J. M., Hanford, L. E., Li, Q., Monzon, F. A., Parks, W. C., and Oury, T. D. (2006). Matrix Metalloproteinases Promote Inflammation and Fibrosis in Asbestos-Induced Lung Injury in Mice. Am. J. Respir. Cell Mol. Biol. 35, 289-297.
Tanaka, H., Kono, E., Tran, C. P., Miyazaki, H., Yamashiro, J., Shimomura, T., Fazli, L., Wada, R., Huang, J., Vessella, R. L., An, J., Horvath, S., Gleave, M., Rettig, M. B., Wainberg, Z. A., and Reiter, R. E. (2010). Monoclonal antibody targeting of N-Cadherin inhibits prostate cancer growth, metastasis and castration resistance. Nat. Med. 16, 1414-1420.
Thiery, J. P., Acloque, H., Huang, R. Y., and Nieto, M. A. (2009). Epithelial-mesenchymal transitions in development and disease
1. Cell 139, 871-890.
Thrall, R. S., McCormick, J. R., Jack, R. M., McReynolds, R. A., and Ward, P. A. (1979). Bleomycin-induced pulmonary fibrosis in the rat: inhibition by indomethacin
11. Am. J. Pathol. 95, 117-130.
Tomasek, J. J., Gabbiani, G., Hinz, B., Chaponnier, C., and Brown, R. A. (2002a). Myofibroblasts and mechano-regulation of connective tissue remodelling
1. Nat. Rev. Mol. Cell Biol. 3, 349-363.
Tomasek, J. J., Gabbiani, G., Hinz, B., Chaponnier, C., and Brown, R. A. (2002b). Myofibroblasts and mechano-regulation of connective tissue remodelling
24. Nat. Rev. Mol. Cell Biol. 3, 349-363.
Utton, M. A., Eickholt, B., Howell, F. V., Wallis, J., and Doherty, P. (2001). Soluble N-Cadherin stimulates fibroblast growth factor receptor dependent neurite outgrowth and N-Cadherin and the fibroblast growth factor receptor co-cluster in cells
1. J. Neurochem. 76, 1421-1430.
Vittal, R., Horowitz, J. C., Moore, B. B., Zhang, H., Martinez, F. J., Toews, G. B., Standiford, T. J., and Thannickal, V. J. (2005). Modulation of prosurvival signaling in fibroblasts by a protein kinase inhibitor protects against fibrotic tissue injury
4. Am. J. Pathol. 166, 367-375.
Wallerand, H., Cai, Y., Wainberg, Z. A., Garraway, I., Lascombe, I., Nicolle, G., Thiery, J. P., Bittard, H., Radvanyi, F., and Reiter, R. R. (2010). Phospho-Akt pathway activation and inhibition depends on N-Cadherin or phospho-EGFR expression in invasive human bladder cancer cell lines
1. Urol. Oncol. 28, 180-188.
Walter, N., Collard, H. R., and King, T. E., Jr. (2006). Current perspectives on the treatment of idiopathic pulmonary fibrosis
1. Proc. Am. Thorac. Soc. 3, 330-338.
Welch, M. P., Odland, G. F., and Clark, R. A. (1990). Temporal relationships of F-actin bundle formation, collagen and fibronectin matrix assembly, and fibronectin receptor expression to wound contraction
1. J. Cell Biol. 110, 133-145.
Werner, S. and Grose, R. (2003). Regulation of wound healing by growth factors and cytokines
5. Physiol Rev. 83, 835-870.
Williams, E. J., Furness, J., Walsh, F. S., and Doherty, P. (1994). Activation of the FGF receptor underlies neurite outgrowth stimulated by 1:1, N-CAM, and N-Cadherin
1. Neuron 13, 583-594.
Willis, B. C., Liebler, J. M., Luby-Phelps, K., Nicholson, A. G., Crandall, E. D., du Bois, R. M., and Borok, Z. (2005). Induction of epithelial-mesenchymal transition in alveolar epithelial cells by transforming growth factor-beta1: potential role in idiopathic pulmonary fibrosis
1. Am. J. Pathol. 166, 1321-1332.
Wipff, P. J., Rifkin, D. B., Meister, J. J., and Hinz, B. (2007). Myofibroblast contraction activates latent TGF-beta1 from the extracellular matrix. J. Cell Biol. 179, 1311-1323.
Zhang, H. Y. and Phan, S. H. (1999). Inhibition of myofibroblast apoptosis by transforming growth factor beta(1)
1. Am. J. Respir. Cell Mol. Biol. 21, 658-665.
Zhang, K., Rekhter, M. D., Gordon, D., and Phan, S. H. (1994). Myofibroblasts and their role in lung collagen gene expression during pulmonary fibrosis. A combined immunohistochemical and in situ hybridization study. Am. J. Pathol. 145, 114-125.
Ziesche, R., Hofbauer, E., Wittmann, K., Petkov, V., and Block, L. H. (1999). A preliminary study of long-term treatment with interferon gamma-1b and low-dose prednisolone in patients with idiopathic pulmonary fibrosis
1. N. Engl. J. Med. 341, 1264-1269.

1-6. (canceled)

7. A method of treating or preventing fibrosis comprising administering an N-Cadherin antagonist which inhibits or neutralizes the activity of N-Cadherin to a subject in need thereof.

8. The method according to claim 7, wherein the antagonist is an anti-N-Cadherin antibody.

9. The method according to claim 7, wherein the fibrosis is Idiopathic Pulmonary Fibrosis.

10. The method of claim 8, wherein the antibody is formulated with a pharmaceutically acceptable carrier.

11. The method of claim 8, wherein the antibody is co-administered sequentially or simultaneously with pirfenidone or interferons.

12. The method of claim 7, wherein the fibrosis is liver fibrosis.

13. The method of claim 7, wherein the antagonist is a siRNA antagonist.

14. The method of claim 7, wherein the antagonist is ADH-1.

15. A method of inhibiting fibroblast-myofibroblast transition (FMT) or epithelial-mesenchymal transition (EMT) comprising administering an N-Cadherin antagonist which inhibits or neutralizes the activity of N-Cadherin to a subject in need thereof.

16. The method of claim 15, wherein the antagonist is an anti-N-Cadherin antibody.

17. The method of claim 15, wherein the antagonist is a siRNA antagonist.

18. The method of claim 15, wherein the antagonist is ADH-1.

19. The method of claim 15, wherein the fibrosis is Idiopathic Pulmonary Fibrosis.

20. The method of claim 15, wherein the fibrosis is liver fibrosis.

21. The method of claim 16, wherein the antibody is formulated with a pharmaceutically acceptable carrier.

22. The method of claim 16, wherein the antibody is co-administered sequentially or simultaneously with pirfenidone or interferons.
